(19) United States
(12) Patent Application Publication
(10) Pub. No.: US 2026/0026447 A1
(43) Pub. Date: Jan. 29, 2026

(54) INDUCTION OF REPRODUCTIVE DIFFERENTIATION IN VEGETATIVE SOMATIC PLANT CELLS, AND METHODS AND CELLS THEREOF

(71) Applicant: UNIVERSITY OF VERMONT AND STATE AGRICULTURAL COLLEGE, Burlington, VT (US)

(72) Inventors: Philip M. LINTILHAC, Shelburne, VT (US); Matthew GRASSO, Milton, VT (US)

(21) Appl. No.: 18/994,248

(22) PCT Filed: Jul. 17, 2023
(86) PCT No.: PCT/US23/27943

§ 371 Date: Jan. 14, 2025

Related U.S. Application Data
(60) Provisional application No. 63/389,564, filed on Jul. 15, 2022.

(51) Int. Cl. A01H 4/00; C12N 5/00; C12N 5/04

(52) U.S. Cl. CPC A01H 4/006; A01H 4/008; C12N 5/0012; C12N 5/04

(57) ABSTRACT

The present disclosure relates to an in vitro method of inducing reproductive differentiation in a vegetative somatic plant cell. This method involves applying mechanical stress and directional force to a vegetative somatic plant cell encapsulated in a polymer material to induce reproductive differentiation in the somatic plant cell. Also disclosed are plant cells and methods and cells thereof.

This application claims the benefit of U.S. Provisional Patent Application Ser. No. 63/389,564, filed Jul. 15, 2022, which is hereby incorporated by reference in its entirety.

### FIELD

The present disclosure relates to the induction of reproductive differentiation in vegetative somatic plant cells and methods and cells thereof.

### BACKGROUND

Sexual reproduction in multi-cellular organisms entails generation of meiotically competent germ cells within a somatic body. While most animals exhibit continuous production from stem cells specified during embryogenesis, angiosperms (flowering plants) are strictly vegetative until intrinsic and environmental cues trigger flowering. In animals, eggs and sperm are pre-formed and can be harvested at any time in the life cycle, for in vitro fertilization for instance, but in plants they arise de novo, appearing suddenly without visible precursors of any kind. There is no existing technology that can be used to manipulate the sexual cycle in plants by inducing the formation of eggs and sperm on demand.

All major crop plants are started from seed. Seed production itself begins with the fusion of an egg and a sperm, whose precursors differentiate in a natural structure called a sporangium, common to all land plants. But there is an absence of any understanding as to how the sporangium promotes the differentiation of eggs and sperm. Within anther and carpel primordia, indeterminate floral progenitor cells differentiate as pre-meiotic archesporial (AR) cells and somatic parietal cells, but the morphogenetic mechanisms responsible remain unclear. The nature of the somatic to germinal induction signal, and the degree to which it is under developmental or physiological control, has been a botanical mystery.

The differentiation of the germ-line in plants is therefore enabled and initiated by some factor or factors capable of specifying which cells, among many identical cells, will differentiate into the initial germ-line and thereafter continue through meiosis and gametogenesis to produce the eggs and sperm. The factors which enable this transition from vegetative to reproductive growth have largely been unknown.

Reproductive differentiation has been assumed to be controlled by diffusible substances, such as hormones or other chemical species (morphogens), causing local concentrations or depletions which trigger the initial stages of reproductive differentiation. These concentration-based mechanisms have proved insupportable, being insufficiently specific to specify single cells, or small clusters of cells, in a mass of otherwise genetically identical somatic cells. Concentration-based mechanisms are also extremely sensitive to natural environmental fluctuations in temperature and other atmospheric fluctuations and therefore generally incapable of providing the spatial and temporal specificity necessary for the precise specification of germ-line precursor cells.

A proposed method of altering the number of archesporial cells in a developing anther of a plant was described in U.S. Patent Application Publication No. 20140359897 to Kelliher and Walbot. This method involved exposing the anther to redox-modulatory conditions prior to differentiation of germline cells in the anther, thereby changing the redox potential of cells in the anther and altering the number of archesporial cells in the anther. Such redox-modulatory conditions refer to the conditions that increase the amount of reactive oxygen species in a cell relative to the same type of cell that is grown under equivalent conditions in air, i.e., the earth's atmosphere, at ground level. Redox modulator conditions were created by exposing a developing anther to hypoxic conditions (e.g., an environment containing less than 1% oxygen), by contacting a developing anther with a redox-modulatory compound, e.g., a reducing agent or oxidizing agent, at a concentration that alters the amount of reactive oxygen species in the cells of the anther. It was thought that this method could be employed to increase or decrease the number of archesporial cells in a developing anther, but the method proved unsuccessful.

The present disclosure is directed to overcoming the above-mentioned deficiencies in the art.

### SUMMARY

One aspect of the present disclosure relates to an in vitro method of inducing reproductive differentiation in a vegetative somatic plant cell. This method involves applying mechanical stress and directional force to a vegetative somatic plant cell encapsulated in a polymer material to induce reproductive differentiation in the somatic plant cell.

Another aspect of the present disclosure relates to a reproductive, germ-line plant cell produced by the in vitro method of inducing reproductive differentiation in a vegetative somatic plant cell described herein.

A further aspect of the present disclosure relates to a haploid plant cell produced by the in vitro method of inducing reproductive differentiation in a vegetative somatic plant cell described herein.

Another aspect of the present disclosure relates to a plant gamete produced by the in vitro method of inducing reproductive differentiation in a vegetative somatic plant cell described herein.

A further aspect of the present disclosure relates to a method of breeding a plant. This method involves uniting a plant gamete produced by the in vitro method of inducing reproductive differentiation in a vegetative somatic plant cell described herein with another plant gamete to form a zygote.

Another aspect of the present disclosure relates to a plant seed produced from the method of breeding a plant described herein, which involves uniting a plant gamete produced by the in vitro method of inducing reproductive differentiation in a vegetative somatic plant cell described herein with another plant gamete to form a zygote.

A further aspect of the present disclosure relates to a plant or germplasm produced from the method of breeding a plant described herein, which involves uniting a plant gamete produced by the in vitro method of inducing reproductive differentiation in a vegetative somatic plant cell described herein with another plant gamete to form a zygote.

Another aspect of the present disclosure relates to a polymer microsphere comprising a polymer material comprising an encapsulated living meiotic germ-line plant cell.

A further aspect of the present application relates to a method of double encapsulating a plant protoplast. This method involves encapsulating a protoplast with agarose (or any other suitable polysaccharide or polymer) to form an agarose microsphere comprising the protoplast; encapsulating the agarose microsphere in alginate methacrylate (or any other suitable polymer) to form an encapsulated agarose microsphere, where the protoplast is encapsulated in both the agarose and the alginate methacrylate.

The present disclosure relates to reproducing the key features of the land plant sporangium to enable the engineered production of sexual gametes (eggs and sperm) in plant cells. Without being bound by theory, it is believed that the sporangium focuses naturally occurring forces produced by growing cells such that a centrally located isotropic point creates a biological singularity that serves to induce the differentiation of the pre-meiotic sexual cells.

The present disclosure comprises a novel understanding of the biomechanical factors surrounding reproductive differentiation in plants, embodied in a methodology that replicates the natural structures, known as sporangia, where reproductive differentiation occurs in plants.

The mechanism described in the methods of the present disclosure presupposes the involvement of naturally occurring mechanical stress, and directional force transmission through precisely shaped physical structures, such that spatially focused mechanical stress fields converge on a single cell, or a small cluster of cells, in a way that irreversibly distinguishes them from their somatic neighbors. The involvement of stress-mechanical elicitors has been poorly studied because of the lack of appropriate methods capable of isolating and manipulating stress-mechanical variables in living plant tissues and cells.

The methods described herein provide a way of precisely manipulating the stress-mechanical environment of specific cells at specific locations, providing a unique and localized eliciting signal that is instantaneous, spatially precise, and insensitive to environmental fluctuations. Namely, described herein are methods of encapsulating protoplasts such that mechanical stress and directional force can be applied to the encapsulated protoplasts in a way to induce reproductive differentiation in a somatic plant cell.

The usefulness of the methods described herein lies in the ability to manipulate the origin of the germ-line in plants, thus enabling the initiation of the germ-line and its sexually competent eggs and sperm in plants that commonly can only be propagated by cuttings or other methods of asexual propagation. Furthermore, the ability to initiate haploidization, meaning the reduction in chromosome number from diploid to haploid, and other genetic and chromosomal manipulations, creates a powerful tool for plant breeders and their methods, enabling them to accomplish genetic manipulations that are not currently possible, such as increasing the number of archesporial cells capable of gametogenesis, and improving the efficiency of sexual reproduction in important crop plants.

Commonly available plant tissue-culture methods are used to produce single, rapidly growing plant cells in sterile culture, removing their native cell walls using combinations of carbohydrate-digesting enzymes to produce living, spherical plant cells known as protoplasts. Droplet-microfluidics is then used to rapidly encapsulate the living protoplasts in micro-spheres or micro-beads of precisely engineered hydrogel materials, which are biocompatible. The micro-beads are permeable to oxygen and necessary nutrients to enable the living protoplasts to survive and grow within the imposed constraints of the micro-bead. The micro-beads are precisely constructed using methods of advanced hydrogel engineering to reconstruct the stress-mechanical environment of natural plant sporangia, focusing internally generated mechanical stress fields on a single cell, or a small cluster of cells, thereby producing precise, spatially focused eliciting signals that initiate the differentiation of the first germ-line cells.

### BRIEF DESCRIPTION OF THE DRAWINGS

FIG. 1 is a schematic illustration of one embodiments of a droplet encapsulation system. One syringe pump flows mineral oil +2% Span 80 through the droplet chip at a flow rate of 1000 μl/hour. Another syringe pump flows protoplasts through the droplet chip at a rate of 180 μl/hour. Agarose mixed at 1.5% in PWS1 is stored in a sealed reservoir and is maintained at a temperature of 35° C. A pressure pump maintains the agarose reservoir at 210 mBar causing agarose to flow through the microchip. The agarose tubing is 10.5 inches long with an inner diameter of 500 μm. Liquid agarose microdroplets exit the microdroplet chip into the outlet tubing where they are carried through an ice water bath causing the agarose microspheres to solidify. Agarose microspheres containing living protoplasts are separated from mineral oil by centrifugation into PWS1. They are then pelleted by centrifugation and transferred to PCM after removal of the PWS1 supernatant.

FIG. 2 is a schematic illustration of one embodiment of an alginate methacrylate double encapsulation system. Two syringe pumps control the flow of mineral oil supplemented with 2% Span 80 and alginate methacrylate solution with agarose encapsulated protoplasts mixed in respectively. The flow rates of the syringe pumps are set at a 10:1 ratio of mineral oil to alginate solution. Microspheres exit the microdroplet chip and flow into a mineral oil+2% span 80 bath with 100 mM CaCl₂ suspended by a magnetic stir bar where they partially crosslink.

FIG. 3 shows photographic images of protoplasts regenerating in agarose microspheres 2, 4, and 6 days after their cell wall digestion and encapsulation in agarose microspheres. The top row shows differential interference contrast images of regenerating protoplasts. The middle row shows CMT organization with mCherry:TUB fluorescence. The bottom row shows cell wall regeneration with calcofluor white cell wall fluorescence stain.

FIG. 4 shows a photographic image of a living plant protoplast showing green due to the presence of the cell viability stain fluorescein diacetate. Labels show the protoplasts is double encapsulated in a layer of both agarose and alginate methacrylate.

FIG. 5 is a photograph image of a fern sporangium with a central cell being specified as a meiocyte. Around this central cell are multiple distinct rings that arise during the development of the sporangium. Replicating this structure in vitro requires a cell culture system with distinct and tunable layers of material around the cells of interest.

### DETAILED DESCRIPTION

Disclosed herein are methods of encapsulating plant cells/protoplasts, methods of inducing reproductive differentiation in vegetative somatic plant cells, and cells and plants produced therefrom.

Unless defined otherwise herein, all technical and scientific terms used herein have the same meaning as commonly understood by one of ordinary skill in the art to which this disclosure belongs. Although any methods and materials similar or equivalent to those described herein can be used in the practice or testing of the present disclosure, exemplary methods and materials are described.

As used herein, the term “archesporial cell” refers to a cell in an anther primordium from which the microsporocytes of a flowering plant develop. Archesporial cells from a variety of different model monocot and dicot species are described in, e.g., Raghavan, V., “mRNAs and a Cloned Histone Gene are Differentially Expressed During Anther and Pollen Development in Rice,” J. Cell Sci. 92:217-29 (1989) (rice); Sheridan et al., “The mac1 Gene: Controlling the Commitment to the Meiotic Pathway in Maize,” Genetics 142:1009-20 (1996) (maize), Sheridan et al., “The mac1 Mutation Alters the Developmental Fate of the Hypodermal Cells and Their Cellular Progeny in the Maize Anther,” Genetics 153:933-41 (1999) (maize); Feng & Dickinson, “Tapetal Cell Fate, Lineage and Proliferation in the Arabidopsis Anther,” Development 137:2409-1 (2010) (Arabidopsis); Ma et al., “Transcriptome Profiling of Maize Anthers Using Genetic Ablation to Analyze Pre-meiotic and Tapetal Cell Types,” Plant J. 50:637-48 (2007) (maize); and Cnudde et al., “Changes in Gene Expression During Male Meiosis in Petunia Hybrida,” Chromosome Res. 14:919-32 (2006) (petunia), which are hereby incorporated by reference in their entirety.

As used herein, the term “prior to differentiation of germline cells” refers to a stage in anther development after the stamen primordia have been initiated from a meristem and prior to the production of meiotically competent germ cells within a somatic body. This stage is considered to be early in anther development.

One aspect of the present disclosure is directed to an in vitro method of inducing reproductive differentiation in a vegetative somatic plant cell. This method involves applying mechanical stress and directional force to a vegetative somatic plant cell encapsulated in a polymer material to induce reproductive differentiation in the somatic plant cell.

As used herein, the term “plant cell” includes cells, protoplasts, cell tissue cultures from which plants can be regenerated, calli, clumps, and cells that are intact in plants or parts of plants including, but not limited to seeds, leaves, stems, roots, vegetative buds, floral buds, meristems, embryos, hypocotyls, cotyledons, endosperm, sepals, petals, pistils, carpels, stamens, anthers, microspores, pollen, pollen tubes, ovules, nucellar tissue, ovaries, and other plant tissue or cells.

The term “protoplast” means the entire viable contents of a living cell without a cell wall (i.e., the cell wall has been removed).

Polymer materials suitable for encapsulating plant cells according to methods described herein include various known biocompatible polymers. Such polymers include, for example and without limitation, agarose, disulfide crosslinked polyacrylamide, alginate, polyvinyl alcohol, PEG-diacrylate, PEG-acrylate/thiol, PEG-azide/alkyne, other acrylates, chitosan, hyaluronic acid, collagen, fibrin, gelatin, and elastin.

In some embodiments, the polymer material is sterile.

In some embodiments, the polymer material encapsulating the plant cell according to methods of the present disclosure is in the form of a spherical droplet.

The encapsulation of plant cells in microbeads is described in Grasso & Lintilhac, “Microbead Encapsulation of Living Plant Protoplasts: A New Tool for the Handling of Single Plant Cells,” Appl. Plant Sci. 4(5):1500140 (2016), which is hereby incorporated by reference in its entirety. Droplet microfluidic systems are capable of rapidly and efficiently capturing large numbers of individual plant protoplasts in precisely sized spherical hydrogel beads. Specifically, living Nicotiana tabacum cv. BY-2 protoplasts were encapsulated in agarose microspheres (Grasso & Lintilhac, “Microbead Encapsulation of Living Plant Protoplasts: A New Tool for the Handling of Single Plant Cells,” Appl. Plant Sci. 4(5):1500140 (2016), which is hereby incorporated by reference in its entirety). Improvements to encapsulation methods are described below.

In some embodiments, the encapsulating polymer material comprises agarose, or any other sterile, biocompatible polymer.

The methods of the present disclosure combine the emerging technologies of droplet microfluidics with advanced hydrogel materials to create structural microspheres containing living plant cells.

As used herein, a “hydrogel” is a substance formed when a non-toxic polymer (natural or synthetic) is set or solidified to create a three-dimensional open-lattice structure that entraps molecules of water or other solution to form a gel. The solidification can occur, e.g., by ionic bonding, aggregation, coagulation, hydrophobic interactions, or covalent cross-linking. Hydrogels can rapidly solidify to keep the cells evenly suspended within a mold (or around or within another solidified gel) until the gel solidifies. Hydrogels can also be biocompatible, e.g., not toxic to cells suspended in the hydrogel. Any suitable hydrogel or other material can be used in methods described herein. Suitable hydrogel examples include, but are not limited to: (1) hydrogels cross-linked by ions, e.g., sodium alginate; (2) temperature dependent hydrogels that solidify or set at body temperature, e.g., PLURONICS™; (3) hydrogels set by exposure to either visible or ultraviolet light, e.g., polyethylene glycol polylactic acid copolymers with acrylate end groups; and (4) hydrogels that are set or solidified upon a change in pH, e.g., TETRONICS™ Examples of materials that can be used to form these different hydrogels include polysaccharides such as alginate, polyphosphazenes, and polyacrylates, which are crosslinked ionically, or block copolymers such as PLURONICS™ (also known as POLOXAMERS™), which are poly (oxyethylene)-poly (oxypropylene) block polymers solidified by changes in temperature, or TETRONICS™ (also known as POLOXAMINES™), which are poly (oxyethylene)-poly (oxypropylene) block polymers of ethylene diamine solidified by changes in pH.

In some embodiments, and as described in more detail infra, combinations of methacrylated hydrogels are used that can be cross-linked specifically to provide physical environments that reproduce the biomechanical environment of the land-plant sporangium.

Specific suitable hydrogel materials include, without limitation, methacrylated dextrans, methacrylated alginates, methacrylated polyethylene glycols, and other synthetic polymers. These polymers can be engineered to shrink, or swell, on demand, resulting in programmable physical deformations being applied to the enclosed living cells, reproducing the biological singularities that occur in the pre-reproductive sporangium, and inducing the developmental shift to reproductive differentiation in competent plant cells.

In some embodiments, plant cells are encapsulated with an engineered “active” polymer(s), that can be programmed or induced (via an external stimulant) to shrink, swell, or otherwise undergo changes in physical behavior that can be used to alter the physical environment of an encapsulated living cell. New advanced hydrogel materials are appearing every year, many of which offer the prospect of manipulating material properties in specific and reproducible ways. Altering the physical environment of living cells encapsulated in a polymer material according to methods of the present disclosure enables application of mechanical stress and/or directional force to the encapsulated cell to induce reproductive differentiation in a somatic plant cell.

Accordingly, in the methods of the present disclosure, plant cells are encapsulated within materials that are capable of inducing or being induced to initiate cellular changes in a plant cell that leads to the differentiation of reproductive cells in plants.

In some embodiments, the encapsulating hydrogel polymer(s) creates precisely localized isotropic singularities that are capable of inducing reproductive differentiation leading to meiosis and the subsequent formation of eggs and sperm from somatic plant cells.

In some embodiments, the encapsulating polymer material forms a spherical droplet around a plant cell or protoplast.

In some embodiments, the spherical droplet is solidified by gelation or by covalent chemical or ionic crosslinking to form a spherical microsphere.

In some embodiments, the polymer-encapsulated plant cells are engineered to form an artificially constituted plant sporangium, the characteristic structure that initiates reproductive differentiation in all land plants.

In some embodiments, polymer encapsulation of plant cells described herein can be used to maintain plant cells in a spatially unpolarized state.

In some embodiments, polymer encapsulation of plant cells can be used to create localized stress-mechanically isotropic singularities in groups of cultured plant cells. Applying mechanical stress and/or directional force on an encapsulated plant cell, such as a protoplast, may be carried out, according to some embodiments, by inducing changes in physical properties of the polymer material.

In some embodiments, the polymer material in which a plant cell is encapsulated according to methods described herein, is physically responsive to external signals or forces to create the mechanical stress and/or directional force applied to an encapsulated plant cell.

In some embodiments, the polymer material is capable of shrink/swell movement to create mechanical stress and direction force on an encapsulated plant cell.

In some embodiments, mechanical stress and/or directional force become focused to comprise isotropic tension and/or compression applied to an encapsulated plant cell.

In some embodiments, the directionally focused forces converge to form an isotropic singularity where all directionality is lost.

In some embodiments, the use of shrink/swell hydrogel polymers are used to manipulate the stress-mechanical environment of encapsulated plant cells. For example, the use of shrink/swell hydrogel polymers can be used to create isotropic tension or compression on encapsulated plant cells according to the methods of the present disclosure. In addition, or alternatively, the use of shrink-swell hydrogel polymers can be used to select individual cells based on their location at the isotropic point in spherical microspheres containing living plant cells.

In some embodiments, applying mechanical stress and/or direction force to an encapsulated plant cell is insensitive, or unresponsive, to environmental fluctuations.

In some embodiments, the plant cell is encapsulated in multiple (more than one) layers of polymer, each of which may have the same or different characteristics, to create a multilayered microsphere encapsulating the plant cell.

In some embodiments, a single plant cell, specifically a protoplast, is encapsulated in a polymer to induce reproductive differentiation in the plant cell.

In some embodiments, more than one plant cell, specifically more than one protoplast, is encapsulated in a polymer to induce reproductive differentiation in at least one or more of the encapsulated plant cells. According to some embodiments, the vegetative somatic plant cell is one of a central cluster of cells, and methods described herein are carried out to induce reproductive differentiation in the cells of the cluster.

In some embodiments, the plant cell(s) is encapsulated in two polymer layers, thus forming a double-layered microsphere, or any shape other than a microsphere.

Another aspect of the present disclosure relates to a reproductive, germ-line plant cell produced by the in vitro method of inducing reproductive differentiation in a vegetative somatic plant cell described herein.

According to this aspect, the present disclosure relates to reproductive plant cells, or gametes, produced by methods disclosed herein. In other words, the reproductive plant cells were vegetative somatic plant cells until encapsulated and induced to reproductive differentiation according to methods described herein.

In some embodiments, such cells, which have been induced to reproductive differentiation according to methods described herein, are haploid cells, gametes, and/or cells capable of being united with another gamete (produced according to methods described herein or harvested from a plant or derived from another method or source) to form a zygote.

A further aspect of the present disclosure relates to a haploid plant cell produced by the in vitro method of inducing reproductive differentiation in a vegetative somatic plant cell described herein.

A “haploid” plant cell refers to a plant cell with one set (n) of chromosomes. Generally, the ploidy level of a genome relates to the number of chromosome sets that are present within the nucleus of the cell. The ploidy level can vary depending upon the type of cells and/or source of cells that make up the organism. The haploid number (n) is an indication that only one set of chromosomes are present within the organism. The dihaploid or diploid number (2n) is an indication that two sets of chromosomes are present within the organism. It is common for some organisms, especially plant species, to contain even greater numbers of sets of chromosomes, e.g., triploid (3n), tetraploid (4n), pentaploid (5n), and hexaploid (6n). Such examples of increased sets of chromosomes, e.g., triploid or greater, are generally known as polyploids.

Typically, the diploid (2n) multicellular stage alternates with a haploid (n) multicellular stage throughout the life cycle of an organism. The haploid (n) stage of the life cycle of an organism is regarded as the gametophytic stage, e.g., gamete producing. Comparatively, the diploid (2n) stage of the life cycle of an organism is regarded as the sporophytic stage, e.g., spore producing. During the sporophytic stage, the organism produces microspores (i.e., spores) that are haploid (n) via a process called meiosis. The resulting microspores can produce gametes, e.g., sperm nuclei that fuse with other gametes, e.g., egg nuclei, produced by megaspores to generate a diploid zygote during fertilization.

In flowering plants the microspores are produced in male reproductive organs. The male reproductive organs are known as anthers. The anthers produce haploid microspores which mature into pollen containing sperm nuclei. The pollen represents the beginning of a short-lived male gametophytic phase of a plant's life cycle during which two sperm nuclei are delivered to the embryo sac of the ovule for double fertilization and subsequent embryo and endosperm formation. Although this stage of a higher plant's life cycle typically involves only a few cell divisions, under certain experimental conditions, microspores can be induced to undergo an altered development, leading to the production of embryo-like structures without an intervening fertilization. As such, these embryo-like structures are haploid (n). This process, referred to as androgenesis, is the biological basis for the in vitro technique known as anther or microspore culture.

Haploid cells can be treated with a chromosome doubling agent, and homozygous plants can be regenerated from haploid cells by contacting the haploid cells, such as haploid embryo cells, with chromosome doubling agents.

Another aspect of the present disclosure relates to a plant gamete produced by the in vitro method of inducing reproductive differentiation in a vegetative somatic plant cell described herein.

As used herein the term “gamete” refers to both male and female reproductive plant cells, tissue, or organs including the anther and ovary (i.e., organs producing pollen and ovules, respectively).

A further aspect of the present disclosure relates to a method of breeding a plant. This method involves uniting a plant gamete produced by the in vitro method of inducing reproductive differentiation in a vegetative somatic plant cell described herein with another plant gamete to form a zygote.

Another aspect of the present disclosure relates to a plant seed produced from the method of breeding a plant described herein, which involves uniting a plant gamete produced by the in vitro method of inducing reproductive differentiation in a vegetative somatic plant cell described herein with another plant gamete to form a zygote.

A further aspect of the present disclosure relates to a plant or germplasm produced from the method of breeding a plant described herein, which involves uniting a plant gamete produced by the in vitro method of inducing reproductive differentiation in a vegetative somatic plant cell described herein with another plant gamete to form a zygote.

Another aspect of the present disclosure relates to a polymer microsphere comprising a polymer material comprising an encapsulated living meiotic germ-line plant cell.

A further aspect of the present application relates to a method of double encapsulating a plant protoplast. This method involves encapsulating a protoplast with agarose to form an agarose microsphere comprising the protoplast; encapsulating the agarose microsphere in alginate methacrylate to form an encapsulated agarose microsphere, where the protoplast is encapsulated in both the agarose and the alginate methacrylate.

Methods of the present disclosure involve encapsulating plant cells, such as plant protoplasts, to create an environment where physical forces resembling the physical forces of the sporangium of a plant are applied to cells in vitro to induce reproductive differentiation in the cells. According to some embodiments, encapsulation of a plant cell involves double encapsulation, such as where an encapsulated plant cell is further encapsulated in a polymer material (the same or different) to form a structure that resembles a plant sporangium.

As discussed in the Examples infra, plant cell encapsulation has been used to encapsulate plant cells in a single layer encapsulation. But methods of double encapsulation described herein are unique to the methods of the present disclosure.

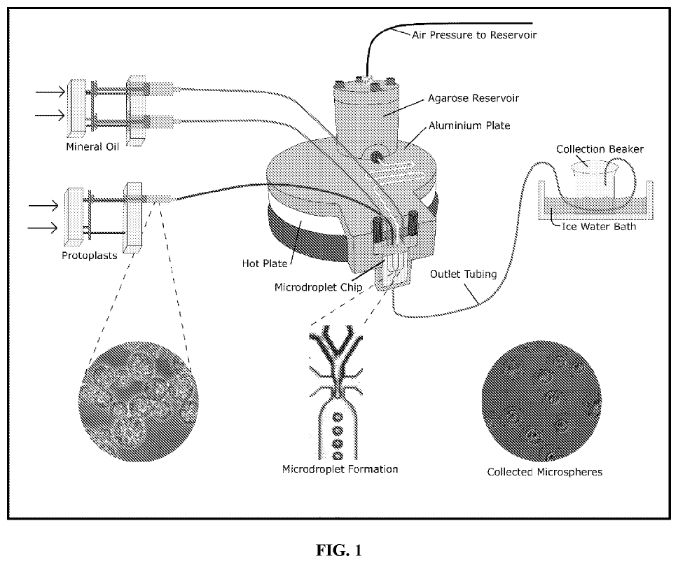

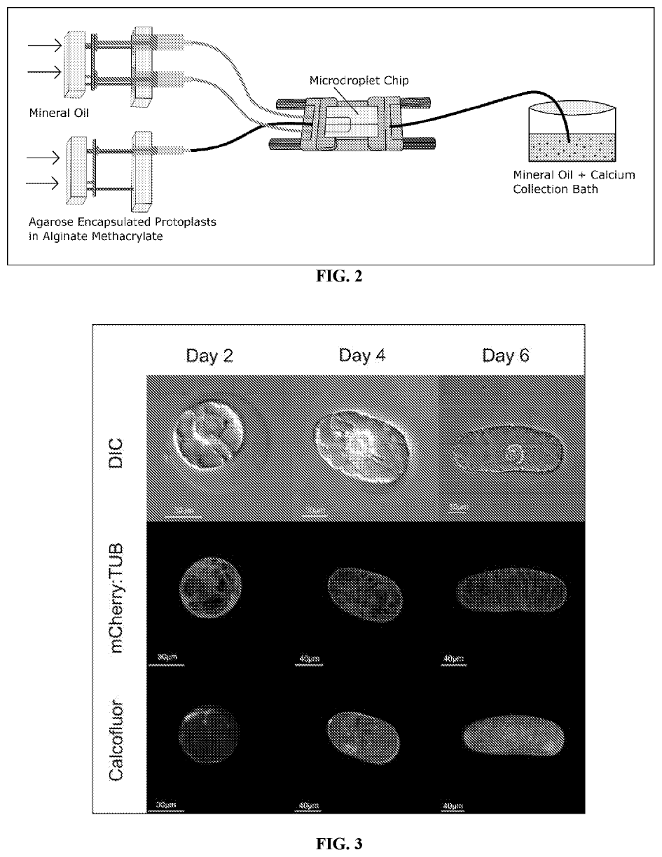

In some embodiments, encapsulating a protoplast comprises passing the protoplast and a polymer material (e.g., agarose) through a microfluidic microdroplet generating system to form an encapsulated cell microsphere. Microfluidic microdroplet generating systems are known, and a suitable system is shown in FIG. 1 and FIG. 2.

In some embodiments, the microfluidic microdroplet generating system comprises a microdroplet chip comprising a first channel for introducing the protoplast; a second channel for introducing the agarose, where the first channel and the second channel combine to form a single microdroplet formation channel; a third channel for introducing mineral oil; and an optional fourth channel for introducing mineral oil, where the third and optional fourth channels combine with the microdroplet formation channel to form the agarose microsphere comprising the protoplast.

As discussed in more detail in the Examples below, the concentration of plant cells passed through a microfluidic microdroplet generating system can be modulated to achieve success in methods described herein. Thus, in some embodiments, methods of encapsulating disclosed herein are carried out with a population of protoplasts at a concentration suitable for efficient encapsulation of a single protoplast at a time. In other words, a solution comprising a population of protoplasts is passed through a microfluidic microdroplet generating system, where the solution comprises a population of protoplasts in a concentration of about 2×10⁶ to 2.25×10⁶ protoplasts/mL, or about 1.8×10⁶ to 2.3×10⁶, or any amount or range therein.

In some embodiments, such as those illustrated in FIG. 1 and FIG. 2, protoplasts are passed through a microdroplet chip contained in the microfluidic microdroplet generating system along with mineral oil and a polysaccharide solution, such as agarose. Microdroplets comprising a single protoplast are formed as the solutions converge. In some embodiments, the formed microdroplets are collected in a collection container or a collection bath. A collection bath may include mineral oil and other ingredients, such as calcium.

Before being encapsulated according to methods described herein, protoplasts may be prepared, according to some embodiments, by contacting a plant cell with an enzyme solution at a suitable osmotic pressure to digest the cell wall to produce a protoplast. In some embodiments, the osmotic pressure is at least 400 mOsM, or an osmotic pressure over 400 mOsM. In some embodiments, contacting with an enzyme solution is carried out at an osmotic pressure of 560 mOsM.

### EXAMPLES

### Example 1—Improvements in Encapsulation Efficiency

The encapsulation of plant cells in microbeads is described in Grasso & Lintilhac, “Microbead Encapsulation of Living Plant Protoplasts: A New Tool for the Handling of Single Plant Cells,” Appl. Plant Sci. 4(5):1500140 (2016), which is hereby incorporated by reference in its entirety. Droplet microfluidic systems are capable of rapidly and efficiently capturing large numbers of individual plant protoplasts in precisely sized spherical hydrogel beads. Specifically, living Nicotiana tabacum cv. BY-2 protoplasts were encapsulated in agarose microspheres (Grasso & Lintilhac, “Microbead Encapsulation of Living Plant Protoplasts: A New Tool for the Handling of Single Plant Cells,” Appl. Plant Sci. 4(5):1500140 (2016), which is hereby incorporated by reference in its entirety). This was accomplished with the use of a microfluidic microdroplet production system that generated a stream of liquid agarose microdroplets in a continuous flow of mineral oil supplemented with the surfactant Span 80. These liquid microdroplets were then cooled causing them to solidify into microspheres. During the production of the liquid microdroplets plant protoplasts were introduced into the liquid agarose causing solidified agarose microspheres to contain living plant protoplasts. These protoplasts were able to regenerate their cell walls and begin to grow again. While this work showed the potential for microencapsulation of living plant cells to develop into a new method for handling them, there were still limitations to the techniques such as low yields and variable cell viability. Furthermore, because the method was being pursued as a way of mechanically manipulating the growth behavior of encapsulated plant cells, materials stronger than agarose to use as an encapsulating matrix were desired. Therefore, follow up work was done to both increase the encapsulation efficiency and identify other materials to use in the encapsulation of plant protoplasts.

The encapsulation efficiency of plant protoplasts in hydrogel microspheres was limited by two main factors. Firstly, variability in the viability of protoplasts following encapsulation in agarose microspheres would lead to many batches of microspheres containing only dead cells a few days following the encapsulation. There was also a tendency for microdroplets of agarose to rejoin with others, also known as droplet coalescence, leading to large globs of agarose material containing many plant cells. Since the goal of the method was to isolate protoplasts in precisely sized microspheres this accumulation of hydrogel material was filtered out as waste. Adjustments to the method were made that improved the viability of protoplasts following encapsulation and increased the overall yield of protoplast containing microspheres by limiting droplet coalescence.

### Improving the Viability of Encapsulated Plant Protoplasts

Low viability of encapsulated plant protoplasts was one of the major limitations in the development of this method. Plant protoplasts are tremendously delicate without their cell walls and as the shearing forces involved in the generation of microdroplets from the stream of agarose are quite aggressive it was suspected protoplasts were being damaged in the process of microdroplet production. Three major adjustments were made that increased cell viability following this step.

First, the osmotic pressure of the enzymatic cell wall digestion solution was increased to near the maximum amount that would not result in protoplast death during cell wall removal. It was increased from 342 mOsM to 560 mOsM, which acted to reduce the overall size of the protoplasts, slightly protecting them from the shearing forces involved in droplet production.

Protoplasts also tended to clump together as they flowed through the tubing that led to the microdroplet chip. This would lead to large clumps of protoplasts pushing through the narrow channels of the microdroplet generation chip causing large amounts of protoplasts to lyse. Reducing the concentration of protoplasts from 5×10⁶ protoplasts/mL to 2-2.25×10⁶ protoplasts/mL allowed protoplasts to flow through the channels without clumping and reduced the number of physical collisions protoplasts experienced in the lead up to microdroplet formation. While this increased the number of agarose microspheres that did not contain a protoplast, it was necessary to improve the consistency of the encapsulation process and prevent the protoplast fluid lines from becoming clogged with cell clumps within the duration of the encapsulation run.

Cell damage leading to low protoplast viability was also caused by over digestion of protoplasts during the enzymatic removal of the plant cell wall. This was in part related to the amount of time the protoplasts spent in the cell wall digestion solution but was also caused by the physical effects of generating protoplasts on a shaker. While shaking protoplasts initially in a digestion can accelerate and make more uniform the removal of cell wall material from suspension culture cells, it is not necessary to maintain protoplasts on the shaker for the entire 3 hours digestion. As protoplasts are released from their cell wall, they become vulnerable and continual shaking can lead to low viability. The updated protoplast generation protocol involves shaking for 60-80 minutes depending on protoplasts release. As most cells are beginning to look spherical regardless of their persisting attachments to neighboring cells they are removed from the shaker and allowed to finish digesting at rest with only intermittent swirling until protoplasts fully release from one another and become isolated. The time required in the shaker to achieve mostly spherical cells has been seen to range from 45-80 minutes with a total digestion time ranging from 2-3 hours before protoplasts are isolated. Variation in the time required to release isolated plant protoplasts from suspension tissue is believed to be a product of the variability in the enzyme mixtures but may also be related to variation in the amount of cell wall found in suspension culture cells.

### Increasing Overall Yield of Protoplast Containing Agarose Microspheres

Droplet coalescence, or microdroplets fusing together to form larger clumps of hydrogel material was another key limitation on the encapsulation efficiency of the method. While unwanted globs of agarose caused by coalescence were easily filtered out, they reduced the total yield of agarose microspheres containing viable plant protoplasts. Most droplet coalescence occurred as microdroplets exited the microchip into the oil collection bath. There they would coalesce before they could be gelled. To minimize droplet coalescence, agarose microspheres were more rapidly cooled following formation. A modification was made to the microdroplet generation system that introduced an outlet tube. While carried in oil flow through the narrow diameter channels, the microdroplets have a low rate of coalescence and therefore by submerging outlet tubing in ice water microdroplets were gelled in flow and did not coalesce while accumulating in the collection bath. This was a very effective modification to reduce droplet coalescence.

### Modification of Agarose Microspheres and the Identification of Alternative Hydrogel Materials

Agarose is a staple material used in the culture of many types of cells and was shown with this work to be suitable for protoplast encapsulation with droplet microfluidics. While it promoted high protoplast viability following encapsulation it was also weak and only able to contain plant cells for 2-4 days before they ultimately burst free from the encapsulating matrix. To manipulate the growth behavior of encapsulated cells alternative materials have been used in place of the agarose.

### Applying a Secondary Coating to Agarose Microspheres

One approach has been to strengthen microspheres through the addition of a polyelectrolyte coating. Polyelectrolytes are molecules of repeating units that contain charged groups. When cationic and anionic polyelectrolytes meet one another their positive and negative charged groups are attracted to one another, and they form a bond. A coating of poly (diallyldimethylammonium chloride) and poly (sodium 4-styrene sulfonate) was successfully applied to agarose microspheres containing plant protoplasts. Solutions of each electrolyte at concentrations of 10 mg/mL were added in quick succession resulting in a small accumulation of polyelectrolyte material on the surface of the agarose microsphere. The thickness of this coating could be increased by repeating this two-step process and washing microspheres in between applications. Protoplasts can survive the application of polyelectrolytes and continue to grow within the modified agarose microsphere. These polyelectrolyte coatings were found to decrease the organization of the microtubule cytoskeleton within growing plant cells and reduce their overall growth.

### Adding Small Amounts of Pectin Into the Microspheres

Pectin is a biologically relevant polymer commonly found in most plant cell walls. It also can form a gel in the presence of divalent cations such as calcium by the ionic bonding of carboxylic acid groups found on its sidechains. Agarose and pectin were prepared together and used to produce the protoplast containing hydrogel microspheres. This combination of hydrogels also produced viable plant protoplasts but did not seem to significantly alter their growth. As more is understood about pectin's role as a signaling molecule it will become more valuable as a tool to manipulate plant growth.

### Using Alginate Methacrylate as a Material

Alginate is a plant-based polysaccharide material that like pectin can form a hydrogel by ionic bonding through divalent cations between carboxylic acid groups on its side chains. These side chains can also be modified with methacrylate groups forming a material called alginate methacrylate. In the presence of free radical ions that can be controllably produced by photoinitiators methacrylate groups will form covalent bonds. This allows alginate methacrylate be stimulated to form a gel in two different ways. While the production of precise alginate methacrylate microspheres that contain living plant cells has been difficult due to the high viscosity of alginate solutions, protoplasts have been successfully encapsulated in irregular pieces of this material.

### Example 2—Method and Apparatus for Inducing the Formation of Eggs and Sperm in Culturable Plant Materials

A sterilizable droplet-forming device and method is disclosed, comprising a glass microfluidic chip with etched channels and a number of computer-controlled fluid pumps which feed living plant cells (protoplasts=cells with the native cell wall removed) carried in a liquid growth medium (channel 1) and a liquid polymer (channel 2), such that the flows of channel 1 and channel 2 mix together to form a viable mix that supports continued growth. See FIG. 1.

The mixed flows of channels 1 and 2 are then intercepted by flows of mineral oil at a microfluidic junction where the surface-tension forces separate the combined flows into micro-droplets, each containing a living plant protoplast.

The droplets, each with its contained living cell, are then solidified, either by temperature (gelation), by catalyzed covalent cross-liking, by ionic cross-linking, or by hydrophobic cross-linking, to form living polymer microspheres comprising an engineered polymer bead containing an embedded living plant cell.

By controlling the mechanical properties of the polymer, the growth and behavior of the embedded cell can be affected such that the developmental fate (life-line) of the cell differentiates from normal vegetative growth and division (mitosis) to the reproductive developmental fate that proceeds through meiosis and the formation of gametes (eggs and sperm).

### Materials and Methods

### Solutions

Suspension Culture Medium (SCM) was prepared in 1000 mL by mixing 30 g Sucrose, 4.33 g MS medium, 0.2 mg 2,4-dichlorophenoxyacetic acid, 1 mg thiamine and 100 mg myo-inositol with the pH adjusted to 5.8 using 0.1 M KOH. After mixing medium was distributed as 100 mL aliquots into 250 mL Erlenmeyer flasks and sterilized by heating in an autoclave for 25 minutes.

Cell Wall Digestion Solution (CWDS) was prepared in 100 mL by adding 9 g mannitol, 0.05 g MgCl2, 0.4 g Cellulase RS, 0.4 g Cellulase R-10, 0.05 g Macerozyme R-10, 0.05 g Pectolyase Y-23, 20 microliters Rohapect UF, 20 microliters Rohapect 10 L, 20 microliters protease inhibitor cocktail, and 0.19% MES to 100 mL of water. The pH was adjusted to 5.8 using 0.1 M KOH and the solution was sterilized using a Rapid-Flow Nalgene Filter Unit.

Protoplast Washing Solution 1 (PWS1) was prepared in 100 mL by adding 9.3 g mannitol, 0.05 g MgCl₂, 0.1 g MES and adjusting the pH to 5.8. The solution was sterilized by heating in an autoclave.

Protoplast Washing Solution 2 (PWS2) was prepared in 100 mL by adding 7.5 g mannitol, 0.05 g MgCl₂, 0.1 g MES and adjusting the pH to 5.8. The solution was sterilized by heating in an autoclave.

Protoplast Culture Medium (PCM) was prepared in 90 mL by adding 3 g sucrose, 0.43 g MS Medium, 0.2 mg/L 2,4-dichlorophenoxyacetic acid, 1 mg/L thiamine, 100 mg/L myo-inositol, 4.5 g mannitol and the pH was adjusted to 5.8 using 0.1 M KOH. The solution was sterilized by heating in an autoclave. After autoclaving 10 mL of Conditioned Medium was added to bring the total volume up to 100 mL.

Conditioned Medium (CM) was collected by centrifuging at 1000 RPM (151 g-force) 50 mL of flask contents from a four-day old liquid suspension culture. The supernatant was run through a 0.45 um filter and stored at −18° C.

Pluronic Solution (PS) was prepared in 100 mL by adding 7.5 g mannitol, 1 g of Pluronic F-68, 5 mM CaCl₂ and adjusting the pH to 5.8 using 0.1 M KOH. The solution was sterilized by heating in an autoclave.

Calcium Solution (CS) was prepared in 100 mL by adding 1.46 g CaCl₂, 4 g mannitol and adjusting the pH to 5.8 using 0.1 M KOH. The solution was sterilized by heating in an autoclave.

Optiprep Solution (OS) was prepared by adding 3 g sucrose 5.5 g mannitol, and 0.05 g MES to 50 mL of water and 25 mL of Optiprep. Next, the pH was adjusted to 5.8 using 0.1 M KOH and the total volume was brought to 100 mL by the addition of water. The solution was sterilized by heating in an autoclave.

### Agarose Encapsulation Procedure

### BY-2 Suspension Culture Maintenance and Cell Harvesting

Nicotiana tabacum c.v. BY-2 (BY-2) liquid suspension cultures were grown in an incubating shaker at 120 RPM and 27° C. in the dark. BY-2 cells were grown in 100 mL of SCM. BY-2 cell suspension cultures were maintained by transferring 1.5 mL of flask contents into a fresh flask of medium every 7 days using sterile technique. BY-2 cell suspension flasks that were four days old since their last subculture were selected for protoplast generation.

### Protoplast Generation

From a 4-day old culture flask 12.5 mL BY-2 cells in SCM were moved to a 50 mL conical centrifuge tube and centrifuged at 1000 (151 g-force) RPM to pellet cells from medium. The supernatant was removed. The BY-2 cell pellet was suspended in 4 mL of CWDS and transferred to a sterile 125 mL Erlenmeyer flask. The flask was placed in an incubating shaker set to 120 RPM at 27° C. for 60 minutes. At this point the flask was moved to an incubator without shaking set to 27° C. The digestion continued with manual swirling every 20 minutes until almost all protoplasts were released from their cell wall.

### Protoplast Cleaning

Protoplasts in CWDS were transferred to a 50 mL conical centrifuge tube and diluted to 15 mL with PWS1. Protoplasts were pelleted by centrifugation at 800 RPM (97 g-force) for 4 minutes and the supernatant was removed. The protoplast pellet was resuspended in 15 mL of PWS1 and pelleted again at 800 RPM (97 g-force). After removal of supernatant this washing step was repeated one more time. Protoplasts were diluted to a concentration of ˜2500 protoplasts/microliter as confirmed by a hemocytometer by the addition of ˜1400 microliters of PWS1+3% Dextran 150000 and 300 microliters of Optiprep Solution. These protoplasts were then run through a sterile 95 micrometer nylon filter to remove large debris and then loaded into a 3 mL syringe for the encapsulation procedure.

### Agarose Microencapsulation Procedure

Plant protoplasts were encapsulated in agarose microspheres using a microfluidic microdroplet generation system. Agarose was prepared at 1.5% in PWS1 and transferred to the heated agarose reservoir through a 0.44 um syringe filter. Equal volumes of mineral oil supplemented with 2% Span 80 was loaded into two 10 mL syringes. Protoplasts and agarose combine in a microdroplet chip briefly before being broken into a stream of microdroplets by the flow of mineral oil. Liquid agarose microspheres were gelled as they flowed through outlet tubing and were collected in a beaker (FIG. 1).

### Microsphere Cleaning

Mineral oil and protoplasts were transferred from the collection beaker to a 15 mL conical centrifuge tube and 6 mL of PWS1 was added. The tube was centrifuged for 4 minutes at 800 RPM causing microspheres to flow down into the denser aqueous phase. Using a hot needle, the bottom of the centrifuge tube was punctured, and microspheres were drained out fully removing them from the oil. Microspheres were transferred to a new 15 mL conical tube and centrifuged at 800 RPM again to pellet microspheres. The supernatant was removed and Agarose microspheres containing living plant protoplasts were transferred to PCM to rest overnight.

### Alginate Methacrylate Double Encapsulation Procedure

### Preparation of Alginate Methacrylate Solution

Alginate methacrylate was sterilized by taking 0.02 grams of alginate polymer and saturating it with 100% ethyl alcohol in a small petri dish. The petri dish was left in a sterile cabinet until alcohol had fully evaporated. Once dry the 0.02 g of sterile alginate polymer was dissolved in 700 μl of PS by aspirating the alginate methacrylate polymer and the PS between two 3 mL syringes attached by a lure lock connector. To prepare agarose microspheres for mixing with the alginate methacrylate solution first agarose microspheres in PCM were pelleted by centrifugation at 800 RPM and the supernatant was removed. Microspheres were then washed by resuspending the pellet in 10 mL of PWS2 and resting for 15 minutes. At this point the agarose microspheres were pelleted by centrifugation at 800 RPM and the supernatant was removed. To the 700 μl of alginate methacrylate solution 300 μl of pelleted agarose microspheres were mixed by addition to the syringe and gentle aspiration. Once incorporated the syringe was ready for the microencapsulation.

### Alginate Methacrylate Double Encapsulation Procedure

The syringe pumps were set with a 10:1 flow rate ratio of mineral oil to alginate methacrylate with a total flow rate low enough to prevent jetting of the alginate solution through the microdroplet chip junction (FIG. 2). Droplets of alginate methacrylate were collected in a mineral oil bath supplemented with 2% span 80 and 100 mM CaCl₂ thoroughly mixed in. A magnetic stir bar in the mineral oil was moving enough to keep the CaCl₂ suspended in oil. After the encapsulation procedure was complete the collection bath contents were transferred to a 50 ml conical centrifuge tube along with 15 mL of CS and centrifuged at 1000 RPM for 5 minutes to pellet alginate microspheres into the calcium solution. Alginate methacrylate double encapsulation microspheres were drained from the bottom of the tube using a hot needle and pelleted by centrifugation again. The CS supernatant was removed, and microspheres were transferred to PCM.

### Results

### Protoplast Regeneration in Agarose Microspheres

Living protoplasts encapsulated in agarose microspheres began to regenerate their cell walls immediately and two days after encapsulation they began to elongate. Eventually elongating protoplasts burst through their encapsulating agarose microsphere as they began to reenter normal mitotic divisions. Images of cortical microtubule (CMT) organization in regenerating protoplasts showed that the cells are spherical in shape and their CMTs tend to be disorganized with no specific orientation. As cells began to elongate their CMTs tended to orient transverse to the axis of elongation (FIG. 3).

### Alginate Methacrylate Double Encapsulation

Protoplasts regenerating in agarose microspheres can successfully be double encapsulated in a layer of alginate methacrylate using the described method. One day after, the alginate methacrylate double encapsulation living plant protoplasts were found in spherical microspheres surrounded by a double layer of agarose and alginate methacrylate (FIG. 4).

### Discussion

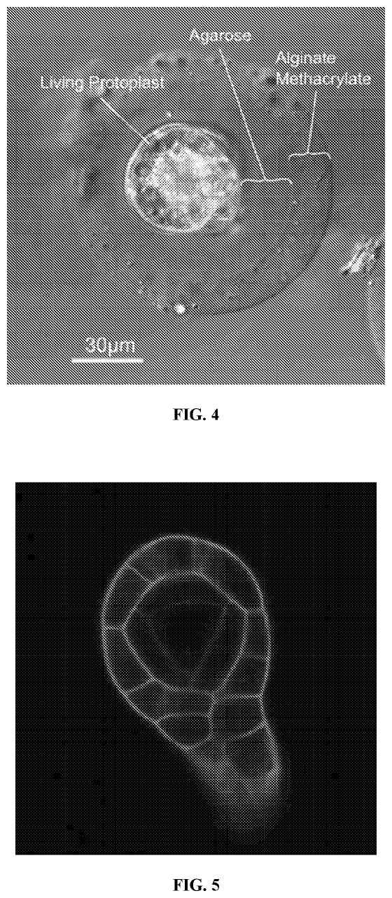

In nature, meiotic plant cells arise in small structures known as sporangia. During the time that cells are specified as meiocytes these structures are circular in cross section with concentric rings focusing out from the central cells (FIG. 5). The double encapsulation method described herein is the beginning of an in vitro culture technique that allows the ability to surround living plant cells with precisely engineered and mechanically tunable multi-layer microenvironments. These microenvironments allow control of mechanics that can influence plant cell growth and differentiation.

### Example 3—Stochasticity and the Limits of Molecular Signaling: The Challenge of Plant Development

Understanding plant development is in part a theoretical exercise that can only succeed if it is based upon a correctly articulated axiomatic framework. In this example, some of the basic assumptions that frame the understanding of plant development are discussed. Foremost among these assumptions is the widely held opinion that the control networks that coordinate and maintain developmental sequencing and morphogenesis in plants is based on the trafficking of informational molecules within cells and between cells. It is proposed that an alternative informational language should be considered, one that is based on the physical relationships between plant cells and tissues that more faithfully reflects the physical and architectural realities of plant tissue and organ growth. The limits of molecular signaling as a stochastic process is discussed, and it is proposed that the iterative and architectural nature of plant growth is more usefully represented by a deterministic model based upon structural, surficial, and stress-mechanical information.

### Introduction

Plant biologists are all familiar with the patterning of plant materials seen in section. To the trained eye these precisely patterned tissues can be interpreted as a frozen history of cell division activity in the growing organ, but it is recognized that the histology of plant tissues and organs is different from the cell arrangements seen in the animal kingdom. To a large extent these differences can be attributed to the fact that animal cells can separate from each other after mitosis and migrate to new locations, effectively obliterating any pattern imposed by cell division itself, while in the plant kingdom cell division erects a permanent wall that can never be moved. This means that plant cells can never migrate; but it also means that permanent structural relationships often cannot be maintained in animals as they are in plants. Movement is fundamental to animal life. Animals need to move to find food, and evolution has provided them with tools suited to that end; highly developed sensory systems, explosive muscle contraction, and the neurological integration that enables animals to interpret their environment in real time. Embryogenesis in the animal kingdom is a narrative of controlled cell movements, reflecting complex molecular signaling systems that are not found to the same extent in the plant kingdom.

But this does not mean that developmental programming is less highly evolved in plants. Far from being the poor cousins of animal development, developmental control networks in the plant kingdom are in fact more architecturally integrated and more spatially deterministic than the complex molecular control networks that evolution has made use of in the animal kingdom.

### The Prevailing Narrative

When development is considered in terms of the cellular processes upon which life is built, it is natural to think in molecular terms. Molecular thinking brings cellularity into the tangible world of things that can be named, manipulated, and even synthesized. Molecules can be thought of as the citizens of a cellular society. They can recognize and exchange information with other molecules. They can archive information for the next generation. Molecules can be visualized. Their structures can be seen in the mind's eye. Markers can be attached to them to follow them around the cell and the genetic templates that they derive from can be edited. So, it is not surprising that developmental differentiation events, and their associated signaling pathways, are thought of as being dependent upon the exchange of molecular information and the migration of key molecular species from one part of a cell to another, or from one part of an organism to another. Hormonal messaging, cellular signal cascades, and the induction of cellular differentiation events are interpreted in terms of a kind of molecular quorum-sensing, meaning that members of the correct molecular species must appear in sufficient number, at the right place, and at the right time to elicit a cellular response.

But molecular signaling is fundamentally a stochastic process (Losick and Desplan, “Stochasticity and Cell Fate,” Science 320:65-6 (2008), which is hereby incorporated by reference in its entirety), which is to say it involves an element of randomness (Bailey, The Elements of Stochastic Processes, Wiley, N.Y., 1964, which is hereby incorporated by reference in its entirety). Moving molecules on the cellular scale is like herding cats. It does not necessarily lead to deterministic outcomes. So, it is reasonable to ask whether there are instances where other informational ecosystems have evolved that can circumvent some of the inherent limitations of molecular signaling. Although current understanding of the programming of plant development and morphogenesis is still rooted in the language of molecular controls, newer work in the development of form and function suggests that plant development is beginning to be seen in a parallel network of structural, mechanical, and surficial relationships that have evolved into a system of geometrically precise and environmentally robust decision-making circuits that are uniquely adapted to the sessile, but architecturally efficient growth-habit of the land plants. It is certainly true that deterministic physical signaling networks are found wherever life has evolved, but in the plant kingdom a case can be made that the structural necessities of plant growth have promoted them as the primary integrator of organogenesis.

### An Evolutionary Perspective

When plants first emerged onto dry land the constraints on growth and form were immediate and dramatic. Buoyant support vanished, and gravitational loads meant that structural adaptations became paramount (Niklas and Spatz, Plant Physics, University of Chicago Press, 2012, which is hereby incorporated by reference in its entirety). Competition for sunlight made it necessary to find a way to raise a vertical axis above the land surface, which in turn necessitated the transport of water to the growing tip.

The evolution of the cellulosic cell wall also enabled plant cells to develop significant turgor pressures which could be used to drive the volumetric growth of cells and organs, and the resulting release of forces caused by local cell expansion could be used to transmit stress-mechanical information over extended distances instantly, accurately, and with no dependence on molecular membership and stochastic molecular signaling. With the evolution of structurally based information new kinds of control circuitry emerged, linking structure, organ topography, and morphogenetic behavior in deterministic feedback loops that support the iterative nature of plant growth (Lintilhac, “The Problem of Morphogenesis: Unscripted Biophysical Control Systems in Plants,” Protoplasma 251:25-36 (2014), which is hereby incorporated by reference in its entirety).

### The Limits of Molecular Signaling

Molecular signaling is beset with all the uncertainties of stochastic processes, particularly where the census number of any signaling molecule is low (Artyomov et al., “Purely Stochastic Binary Decisions in Cell Signaling Models Without Underlying Deterministic Bistabilities,” PNAS 104(48):18959-18963 (2007), which is hereby incorporated by reference in its entirety), and although molecular signals can be highly selective in targeting specific receptors and eliciting specific responses, they require the transport of molecules from one location to another in roughly stoichiometric quantities, regardless of entropic signal degradation and environmental perturbations.

Physical signaling networks, on the other hand, are manifestly deterministic. Force transmission is directional, instantaneous, and spatially predictable. It can effect action at a distance without molecular transport of any kind; and given the strong and permanent mechanical coupling between cells in the plant kingdom, and the ability of growing tissues to generate significant surface-bounded stress fields, it seems reasonable to assume that evolution would have found ways to explore and enlist material behaviors and stress-mechanical relationships (Hernandez-Hermandez et al., “Mechanical Forces as Information: An Integrated Approach to Plant and Animal Development,” in Frontiers in Plant Science 5:1-16, Article 265, 2014, which is hereby incorporated by reference in its entirety) to coordinate cellular proliferation, organic form, and the precise placement of new division walls (Facette et al., “Plane Choice: Coordinating Timing and Orientation of Cell Division During Plant Development,” Current Opinion in Plant Biology 47:47-5 (2019), which is hereby incorporated by reference in its entirety). From this perspective it becomes possible to think of morphogenesis in the plant kingdom as an emergent manifestation of physical and surficial feedback circuits that regenerate themselves without requiring direct genetic scripting (Lintilhac, “The Problem of Morphogenesis: Unscripted Biophysical Control Systems in Plants,” Protoplasma 251:25-36 (2014), which is hereby incorporated by reference in its entirety).

Plant apical meristems are shape generators. But shape is more than just an outcome, it is a controlling variable.

Shapes are contained within surfaces, and free surfaces impose simple rules on the behavior of principal stresses (Heywood, Photoelasticity for Designers, p. 197, Pergamon Press, N.Y., 1969, which is hereby incorporated by reference in its entirety). Surfaces can act as waveguides that channel force transmission (Frocht, Photoelasticity, Volume I., pp. 215-225, Wiley, N.Y., 1962, which is hereby incorporated by reference in its entirety), effectively molding the stress fields that build up under the surface. The information in these stress fields then becomes available to dividing meristematic cells and can be used to determine the orientation of new division walls.

Physical and material feedbacks are demonstrably critical during plant development. They integrate a variety of material behaviors from the nano-structural level to the level of whole tissues and organs, providing robust, instantaneous, and highly directional signals that can be interpreted and acted upon at the cell and tissue levels, resulting in the precise division wall orientations that we see everywhere in embryonic plant tissues (Jackson et al., “Global Topological Order Emerges Through Local Mechanical Control of Cell Divisions in the Arabidopsis Shoot Meristem,” Cell Systems 8:1-13 (2018), which is hereby incorporated by reference in its entirety).

Nonetheless, addressing the deficiencies in the understanding of plant development requires more than just an acknowledgement of the limits of stochastic molecular signaling. It involves visualizing and documenting the networks of physical interaction that control morphogenesis at the tissue and organ levels. While molecular signals can be visualized and followed in various ways, including the use of molecule-specific labels, transmitted force is essentially invisible. There are no fluorescent probes for tension and compression. And at the cellular level we need to understand the nature of the relationship between transmitted force and the positioning of the cell plate during cell division. How does the cell resolve and respond to the forces acting on it?

It has been known for many years that the patterns of division wall placements seen in actively dividing plant tissues reflect the principal stress fields radiating through appropriately configured photoelastic models (Lintilhac and Vesecky, “Stress-induced Alignment of Division Plane in Plant Tissues Grown In Vitro,” Nature 307:363-364 (1984), which is hereby incorporated by reference in its entirety). But new experimental tools are needed that allow simplifying stress-mechanical relationships and isolating critical variables. Experimental systems are needed where single plant cells can be subjected to explicitly defined stress-mechanical inputs (Grasso and Lintilhac, “Microbead Encapsulation of Living Plant Protoplasts: A New Tool for the Handling of Single Plant Cells,” Applications in Plant Sciences 4(5):1500145 (2016), which is hereby incorporated by reference in its entirety), and the resulting cellular behaviors can be more precisely monitored. Ultimately, new and innovative methods are needed that can be interpreted at both the cellular and tissue levels that allow mapping the biomechanical landscape of organogenesis taken as a whole.

Historically, visualizing stress distributions and separating their tensile and compressive components was accomplished with photoelastic stress analysis (Lintilhac, “Differentiation, Organogenesis, and the Tectonics of Cell Wall Orientation. II. Separation of Stresses in a Two-dimensional Model,” Amer. J. Bot. 61(2):135-140 (1974), which is hereby incorporated by reference in its entirety), a modeling technique which has largely been superseded by computer-based finite-element modeling. More recently however, photoelastic analysis has re-emerged in the form of Digital Photoelastic Analysis (Solaguren-Beascoa et al., “Stress-separation Techniques in Photoelasticity: A Review,” J. Strain Analysis 45:1-17 (2009), which is hereby incorporated by reference in its entirety), which combines the visual immediacy that derives from photoelastic rendering with the convenience of digital extraction of principal stress trajectories.

### Conclusion

The physical and biomechanical circuitry that ties all plant development together must be understood in the context of a robust, deterministic progression of morphogenetic changes. The theoretical framework of plant developmental biology may be at a juncture where the multitude of molecular subsystems that define life at the cellular level can be understood within the context of a unifying paradigm, reflecting an ecosystem of deterministic material behaviors that have been conscripted by evolution to generate robust signaling networks that manage development throughout the plant kingdom. Cell and tissue mechanics then becomes the language through which developmental information is transmitted.

### Example 4—Stress-Mechanical Singularity in Sporangial Development

It is a remarkable, if largely unappreciated fact that the initial events leading to the differentiation of haploid gametes in the land plants remain a complete mystery. Whether the result of technical insufficiency, or benign neglect, this deficiency would seem to demand the attention of researchers and students alike. In this example, the primary induction event that opens the path to sexual reproduction in all the land plants is discussed. Never has a mechanism been proposed that adequately explains how ordinary somatic cells, initially undistinguished from their clonal neighbors, can abruptly shift to a developmental fate that commits them to meiosis, haploidization, and gametogenesis.

Crop plants that are propagated from seed, including all cereal grains and sexually propagated flowering plants, depend upon the fusion of haploid gametes during syngamy. All haploid gametes trace their developmental lineage back to this singular and unexplained event that occurs exclusively within the confines of specialized structures called sporangia.

A biomechanical mechanism that is positionally precise, temporally instantaneous, robust in the face of environmental fluctuations, and insulated from the molecular vagaries of direct genetic scripting is proposed.

It may be that by continuing to unravel the tangle of molecular pathways and signaling cascades that carry out the daily functionalities of plant life it is possible to eventually come to a comprehensive understanding of plant development and morphogenesis as it relates to the initiation of the reproductive germ line in plants, but a problem that has proven to be as recalcitrant as this raises the question of whether it is seen correctly to begin with. A new paradigm is needed for understanding control systems in plant development in order to resolve this fundamental enigma.

Plants initiate the sexual cycle by meiotic reduction division and the differentiation of eggs and sperm just as animals do, but the timing of gametogenesis in the land plants is distinct from gametogenesis in the animal kingdom where, with some few exceptions, sexual precursor cells are set aside early in life and are carried along as a “Reserved Germ-Line.” This is evident from the well-known fact that human eggs and sperm exist from infancy and are maintained as a distinct lineage throughout our reproductive lives. In the plant kingdom there is no reserved germ-line. During vegetative growth there is no lineage of cells that leads directly to meiosis and gamete formation. Instead, some mechanism is brought into play that precipitates the specification of a single cell, or a small group of cells, that abruptly diverge from the vegetative developmental growth habit of clonal somatic cells and differentiate to form the germ line, proceeding from there through meiosis and gametogenesis to form eggs and sperm.

This foundational event that accounts for the establishment of the germ line in plants is the single most critical differentiation event in the reproductive life-cycle of all the land plants, and yet there is no working hypotheses to account for it, no feasible mechanisms to query experimentally, and very little ongoing work that attempts to articulate a viable explanatory narrative.

Understanding sexual reproduction in the land plants is a challenge with far-reaching implications, not only for plant breeding and crop science, but for the ability to interpret developmental control systems as a whole. The immediacy of this challenge becomes starkly apparent when it is considered that virtually 100% of land-based food chain depends upon this single, brief, developmental event.

Understanding this transition from vegetative growth to a distinctly reproductive lineage presents a number of experimental obstacles. Differentiation of the meiotic precursor cell(s) is both cryptic and fleeting; a seemingly momentary change in behavior, hidden away in the tiniest and most delicate of plant structures at the very earliest stages of their development. At one moment the growing structure seems to be uniformly vegetative; at the next moment a distinctly different cell type reveals itself as the presumptive archesporium and the complexities of reproductive differentiation ensue.

To review the basic facts, in plants meiosis always happens in specialized structures called sporangia which share certain unifying features. Sporangia are typically organized as volumes of revolution, with a concentrically layered sporangial wall and a central zone, known as the archesporium, where pre-meiotic differentiation occurs. The divisions that lead up to and anticipate reproductive differentiation in the archesporium are typically highly predictable and result in a distinctive and stereotypical patterning of the young sporangium.

To clarify the context within which germ-line initiation proceeds the constraints that it must meet must be defined. Clearly, the most stringent of these is that it must be capable of reliably selecting a single cell embedded within an aggregation of otherwise identical somatic cells. One can think of this as a process of singularization, by means of which the initiating stimulus mechanism can create a robust singularity no larger than a single cell.

Examples of this are found in the ability to create precise single cell localizations throughout the plant kingdom. The most familiar example being in the nucellus (megasporangium) of the flowering plants. In the nucellus, regardless of differences in the details of nucellar anatomy, archesporial specification selects only a single cell, centrally located, which differentiates to become the “megaspore mother cell.”

Flowering plant anther sacs (microsporangia) on the other hand, include a central column of pre-meiotic cells that proceed through meiosis and on to microspore and pollen formation. However, the single-celled archesporium is clearly the most stringently defined in terms of size and location. This means that the initiating stimulus needs to be able to resolve a single cell, precisely and repeatably, regardless of fluctuations in temperature or other environmental disturbances. These spatial constraints are quite severe. Cell dimensions in developing nucelli can be as small as 15 or 20 microns, making it difficult to envision a diffusion-based process or a stochastic molecular morphogen delivery system that can match these spatial and geometric constraints.

These spatial constraints raise fundamental questions about the nature of developmental control systems in plants. Can we really expect to untangle the questions of spatial resolution and informational robustness that are posed by these earliest events in reproductive differentiation by invoking molecular signaling systems that are essentially stochastic in nature, and generated symplastically at the tissue level? What kinds of transport circuitry can reliably focus diffusible molecular species on a single cell, embedded in a population of clonally identical siblings?

The intractability of this particular instance of developmental differentiation, and the inability to visualize an experimental approach to its resolution raises the broader question of whether we are seeing the problem in terms of the wrong paradigm. Are we trying to explain a suite of developmental phenomena in terms of molecular networks that are in fact networked on another level?

Historically, the anatomical and geometrical features of land-plant sporangia have been faithfully recorded, beginning with the work of Bower, Goebel, and others in the 19^{th}century, but questions of developmental control could never be approached systematically until the tools of molecular genetics became available in the middle of the 20^{th} century.

Now another technical deficiency is becoming apparent. We need a new generation of biomechanical and biophysical tools that allow us to dissect the physical and structural control networks that have evolved in plants. These networks, while completely dependent upon the heritable register of the genome, have evolved independently, as an emergent form of structural information that plants have mastered that applies to all aspects of plant growth and morphogenesis. These networks, entraining intrinsic material behaviors in complex feedbacks that reach from the nanostructural level to the level of the whole plant, have the effect of relieving the genome of the need to detail the choreography and timing of individual developmental manipulations.

When the early developmental anatomy of these structures is studied, certain facts stand out as being common to all land plant sporangia. The cells of the pre-meiotic sporangium are initially indistinguishable from each other in every regard except their position. They all look the same—not surprising since they represent a cohort of clonal mitotic derivatives. No single cell can be distinguished from its neighbors as being pre-determined for archesporial differentiation. And, given their clonal history, it cannot be said that any one cell is genetically predisposed to reproductive differentiation. Nevertheless, at some critical moment, within this defined group of precisely arranged sporangial cells, a single cell, or in many cases a small cohort of cells, reveal themselves to be clearly different from their previously identical neighbors. This abrupt change identifies the first visible pre-meiotic archesporial cells, always appearing at the geometrical center of the sporangium at a precise moment in time.

By this line of reasoning, transcription-directed programming can be eliminated as a direct cause of reproductive specification in plants. So, what is it about the structure and anatomy of the sporangium that gives it the ability to specify individual cells and divert them towards meiosis and gametogenesis?

To understand this event in terms that reflect its remarkable consistency throughout the land plants one needs to see beyond the historical view of developmental control systems as molecular response networks. Molecular signal networks are essentially stochastic processes. They depend on being able to adjust populations of molecular species at a particular location in space, and at the correct time in order to elicit a change in behavior of cells locally. All of which must be accomplished while dissipative forces are working to level the peaks and valleys. At a certain scale this becomes an effective signaling strategy, but at the small scale of archesporial determination keeping molecules in or out of a single cell at a particular location becomes problematic, particularly in light of the temperature sensitivity of molecular diffusion.

Physical signaling, on the other hand, does not rely on molecular transport at all. Being intrinsically directional and immune from dissipative tendencies it can be directed to carry out action at a distance instantly. Physical forces can travel through material objects at the speed of sound, and can converge with pin-point accuracy on a singular location in space. Directionalities can be shaped by surfaces and guided to distant targets where they can elicit specific behaviors. Physical force applied to an object is transmitted through the material as mechanical stress, either tensile or compressive, and although the stresses radiating through the material are invisible to the observer they can be modeled and resolved into patterns of principle stress that obey simple rules governing their behavior. It is also known that these stresses obey simple rules with respect to their behavior at surfaces, which can act to channel stresses in strict regard to surface topology. Plant cells are known to be sensitive to these stress directionalities and respond to them with precisely oriented cell-plate orientation.

The hypothesis put forward here is that by using the physical characteristics inherent in all plant tissues, namely, strong mechanical coupling, the ability to transmit directional stress information rapidly and with good spatial resolution, and immunity from diffusional degradation, plants have evolved an ideal signaling system that is capable of selecting individual cells for reproductive differentiation in plants. The role of the sporangium can then be seen as a stress-mechanical lens that focuses the mechanical forces generated by growth on a centrally located region, where the normal directionality of force transmission vanishes, creating what can be described in stress-mechanical terms as an isotropic singularity.

This concept of “singularity” in the context of plant reproductive differentiation acknowledges a unique developmental tool that has emerged in the land plants, reflecting the structural necessities of the land plant growth habit, and the requirement for idiographic specification of the germ line in otherwise uniformly clonal vegetative sporophytes.

But the concept of stress-mechanical singularity in sporangial development is not without its own consequences and difficulties. First among these is the lack of experimental tools that are suited to the task of exploring the underlying mechanisms in detail. While the tools available for molecular studies are remarkably diverse and specific, the tools available for studying force transmission directly at the tissue level are virtually non-existent. Direct tracking of principle stress distributions and trajectories in plant tissues is virtually impossible. Photoelastic methods have been used to visualize stress trajectories in cultured plant materials embedded in polymer embedding media, but the intrinsic birefringence of the plant cell wall makes polarized light methods difficult to apply in plant tissues. Effective modeling can be accomplished using the Finite Element Method (“FEM”) but models are only as good as the information put into them and at these small dimensions the ability to measure cell wall moduli and cell turgor pressures are largely out of reach at present. FEM analysis of stress distributions in continuous and homogeneous solids is relatively straightforward, but plant tissues are more complex. Direct force transmission through multicellular tissue becomes difficult to interpret at the cellular level because it disappears into the realm of turgor pressure differences and cell wall tensile stress. But perhaps the most challenging difficulty lies in the very small size of these structures at the time of germ line specification, making direct manipulation of the physical parameters that govern stress distribution extremely difficult and hard to interpret.

However, if we if we distance ourselves from the trend of explaining all cellular differentiation in terms of molecular signal cascades, and examine the salient facts surrounding the spatial and temporal specificity of the reproductive differentiation, we find that they point to a system of integrated structural and physical signals unique to plants, reflecting evolution's ability to explore alternate ways to assemble an effective circuitry of developmental controls.

Germ line initiation in plants is always keyed to location. The cells that transition to meiosis are always precisely central in the sporangium. This tells us that we are dealing with an inducing signal that is strictly positional. Whether we are dealing with ferns or flowering plants the first distinguishable archesporial cells are without exception confined to the geometric center of the sporangium. There are no examples of pre-meiotic cells arising in the sporangial wall. It can be concluded that germ line determination reflects some stimulus that is positionally determined.

It has become a rule of thumb, when considering developmental control systems, to think of them in molecular terms, with genome transcription and molecular signaling cascades in the foreground, and with the architectural and biomechanical aspect of development being relegated to the background. But in the plant kingdom, it is becoming increasingly clear that physical forces, shaped by structures and surfaces, have moved into the foreground of developmental control, having evolved into self-referential control networks that follow their own internal rules of behavior not scripted directly from the genome. As we begin to unravel these physical control networks (circuits) exquisitely balanced physical behaviors will be shown to have precise structural consequences that can elicit specific biological outcomes that no molecular signaling system can accomplish.

Nevertheless, the link between colligative molecular effects working at the cellular level and the physical behaviors available at the whole organ and tissue levels are important. Molecular populations can prime the biophysical pump by controlling osmotic water-potentials and the turgor pressures that that drive cell enlargement, generating the forces that are transmitted from cell to cell, radiating through multicellular tissues as mechanical stress fields. This is particularly relevant in the case of plant tissues, where cell movement does not occur, and where force transmission is made possible by the strong mechanical coupling of cells at the tissue level.

Growth itself is a highly directional process in plants, controlled by the nanostructural configuration of the wall itself.

Even where cellular pressure differentials are insufficiently precise to locate a single cell among many, the transmitted forces generated by pressure-driven cell enlargement can be shaped by surface topology and channeled at precise locations. In this way geometric accuracy can be elicited from a stochastic molecular process of low precision. In plant systems, where molecular and osmotic relationships can be readily translated into the direct transmission of force from one location to another, and where individual cells maintain unbreakable physical relationships with their neighbors, the merits of physical signaling circuitry become clear.

Plant development is integrated at the physical level. The production of shape and form can only be understood through the confluence of mechanical stress, surface topology, and the material properties of tissue components, all of which can be tuned at the molecular level.

The physical and biochemical aspects of plant growth represent two distinct domains of developmental logic, the first being the domain of direct physical mechanics, and the second being the domain of stochastic molecular processes. While the physical domain cannot exist without the biochemical domain, it is also the only way to understand the momentary structure and architecture of the growing plant.

Some of the simplest sporangia are found in the leptosporangiate ferns. But as in all plants, archesporial specification occurs during the earliest observable stages of sporangial development, when the sporangia are still microscopic, comprising of only a handful of precisely formed polyhedral cells. In the flowering plants separate male and female sporangia are found, each of which anticipate separate meiotic specification events that occur in the unopened flower bud, long before anthesis.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An in vitro method of inducing reproductive differentiation in a vegetative somatic plant cell, said method comprising:
applying mechanical stress and directional force to a vegetative somatic plant cell encapsulated in a polymer material to induce reproductive differentiation in the somatic plant cell.

2. The method according to claim 1, wherein the polymer material comprises agarose.

3. The method according to claim 2, wherein the agarose is in the form of a spherical droplet.

4. The method according to claim 2 or claim 3, wherein the agarose is further encapsulated in alginate.

5. The method according to claim 4, wherein the somatic plant cell is encapsulated in a double-layered microsphere.

6. The method according to anyone of the preceding claims, wherein said applying mechanical stress and directional force is carried out by inducing changes in physical properties of the polymer material.

7. The method according to any one of the preceding claims, wherein the polymer material is physically responsive to external signals or forces to create the mechanical stress and directional force.

8. The method according to any one of the preceding claims, wherein the polymer material is capable of shrink/swell movement to create the mechanical stress and directional force.

9. The method according to any one of the preceding claims, wherein the mechanical stress and directional force comprise isotropic tension and/or compression.

10. The method according to claim 9, wherein the mechanical stress and directional force form an isotropic singularity where all directionality is lost.

11. The method according to any one of the preceding claims, wherein the vegetative somatic plant cell is one of a central cluster of cells, and said method is carried out to induce reproductive differentiation in the cells of the cluster.

12. The method according to any one of the preceding claims, wherein said applying mechanical stress and directional force is insensitive to environmental fluctuations.

13. A reproductive, germ-line plant cell produced by the method of any one of the preceding claims.

14. A haploid plant cell produced by the method of any one of claims 1-12.

15. A plant gamete produced by the method of any one of claims 1-12.

16. A method of breeding a plant, said method comprising:
uniting the plant gamete of claim 15 with another plant gamete to form a zygote.

17. A plant seed produced from the method of claim 16.

18. A plant or germplasm produced from the method of claim 16.

19. A polymer microsphere comprising a polymer material comprising an encapsulated living meiotic germ-line plant cell.

20. The polymer microsphere according to claim 19, wherein the polymer material comprises agarose.

21. The polymer microsphere according to claim 20, wherein the agarose is in the form of a droplet.

22. The polymer microsphere according to claim 20 or claim 21, wherein the agarose is encapsulated in alginate.

23. The method according to claim 22, wherein the encapsulated living meiotic germ-line plant cell comprises a double-layered microsphere.

24. A method of double encapsulating a plant protoplast, said method comprising:
encapsulating a protoplast with agarose to form an agarose microsphere comprising the protoplast;
encapsulating the agarose microsphere in alginate methacrylate to form an encapsulated agarose microsphere, wherein the protoplast is encapsulated in both the agarose and the alginate methacrylate.

25. The method according to claim 24, wherein said encapsulating a protoplast comprises passing the protoplasts and the agarose through a microfluidic microdroplet generating system to form the agarose microsphere.

26. The method according to claim 25, wherein said microfluidic microdroplet generating system comprises:
a microdroplet chip comprising:
a first channel for introducing the protoplast;
a second channel for introducing the agarose, wherein the first channel and the second channel combine to form a single microdroplet formation channel;
a third channel for introducing mineral oil; and
an optional fourth channel for introducing mineral oil, wherein the third and optional fourth channels combine with the microdroplet formation channel to form the agarose microsphere comprising the protoplast.

27. The method according to claim 25 or claim 26, wherein the protoplast is passed through the microfluidic microdroplet generating system in a population of protoplasts at a concentration of about 2 to 2.25×10⁶ protoplasts/mL.

28. The method according to any one of claims 25-27, wherein mineral oil is passed through the microfluidic microdroplet generating system with the protoplast(s) and the agarose.

29. The method according to any one of claims 25-28, wherein the agarose microsphere is collected in a mineral oil bath.

30. The method according to any one of claims 24-29, wherein the protoplast(s) is generated from a method comprising:
contacting a plant cell with an enzyme solution at an osmotic pressure of over 400 mOsM under conditions effective to digest the cell wall to produce the protoplast.

31. The method according to any one of claims 24-30, wherein said contacting is carried out at an osmotic pressure of about 560 mOsM.
